(19) United States
(12) Patent Application Publication
(10) Pub. No.: US 2004/0180368 A1
(43) Pub. Date: Sep. 16, 2004

(54) Method for producing a high density nucleic acid array using activators

(75) Inventor: Glenn H. McGall, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/742,301

(22) Filed: Dec. 18, 2003

Related U.S. Application Data
(60) Provisional application No. 60/436,312, filed on Dec. 23, 2002.

(51) Int. Cl. C12Q 1/68; C12M 1/34; B05D 3/00

(52) U.S. Cl. 435/6; 435/287.2; 427/2.11

(57) ABSTRACT

Methods are provided for fabricating high density oligonucleotide arrays with activators. According to the disclosed methods for preparing high density oligonucleotide arrays on a solid support, the following steps are provided: a) providing a solid support having a surface comprising functional groups; b) attaching an activated nucleotide to a functional group in the presence of an activator; c) repeating the step of attaching an activated nucleotide to form an oligonucleotide array having at least 100 different oligonucleotides/cm².

### PRIORITY CLAIM

This application claims priority of U.S. Provisional Application Serial No. 60/436,312, filed on Dec. 23, 2002 which is incorporated herein by reference for all purposes.

### FIELD OF THE INVENTION

The present invention relates to a method for producing high density oligonucleotide arrays using activators.

### BACKGROUND OF THE INVENTION

High density oligonucleotide arrays on solid substrates have wide ranging applications and are of substantial importance to many industries including, but not limited to, the pharmaceutical, biotechnology and medical industries. For example, the arrays can be used in screening large numbers of molecules for biological activity, e.g., DNA-binding capability and identifying mutations in known sequences.

The present invention provides methods for producing high density nucleic acid arrays using activators.

### SUMMARY OF THE INVENTION

Methods for fabricating high density oligonucleotide array are disclosed. According to the disclosed methods, the following steps are provided: a) providing a solid support having a surface comprising functional groups; b) attaching an activated nucleotide to a functional group in the presence of an activator; c) repeating the step of attaching an activated nucleotide to a functional group to form an oligonucleotide array having at least 100 different oligonucleotides/cm².

Activators which may be employed in the instant invention are tetrazole, DCI, ETT and PCI. Of these ETT and DCI are particularly preferred for use with the present invention. Use of the activators as set forth here for fabrication of oligonucleotides arrays allows the use of less phosphoramidite regent, thus substantially reducing cost.

### DETAILED DESCRIPTION OF THE INVENTION

### DEFINITIONS

The terms “solid substrate” and “solid support” are used interchangeably herein and refer to the bulk, underlying, and core material which can contain additional layers of material. The solid support is a material having a rigid or semi-rigid surface. Such materials preferably take the form of plates or slides, small beads, pellets, disks or other convenient forms, although other forms can also be used. In some embodiments, at least one surface of the substrate is substantially flat. In other embodiments, a roughly spherical shape is preferred. The solid support can be biological, nonbiological, organic, inorganic, or a combination of any of these, existing as particles, beads, strands, precipitates, gels, sheets, tubing, spheres, containers, capillaries, pads, slices, films, plates, slides, etc. The solid support is preferably flat but may take on alternative surface configurations. For example, the solid-support may contain raised or depressed regions on which synthesis takes place. Exemplary supports include, but are not limited to, glass (including controlled-pore glass), polymerized Langmuir Blodgett films, silicone rubber, quartz, latex, polyurethane, silicon and modified silicon, Ge, gallium arsenide, GaP, silicon dioxide, silicon nitride, metals (such as gold, and other derivatizable transition metals, a variety of gels and polymers such as (poly)tetrafluoroethylene, (poly)vinylidendifluoride, polystyrene, polystyrene-divinylbenzene copolymer (e.g., for synthesis of peptides), polycarbonate, and combinations thereof. Other suitable solid support materials will be readily apparent to those of skill in the art. Solid-support base materials are generally resistant to the variety of chemical reaction conditions to which they may be subjected.

The term “oligonucleotide” refers to a polymer having at least two nucleic acid units, preferably at least about 25 nucleic acid units, more preferably at least about 40 nucleic acid units, and most preferably at least about 60 nucleic acid units.

The terms “nucleotide,” “nucleic acid” and “nucleic acid unit” are used interchangeably herein and refer to both natural and unnatural nucleic acids and derivatives thereof.

The term “solid support bound oligonucleotide” refers to an oligonucleotide that is covalently bonded to a solid-support.

The term “linker” means a molecule or group of molecules attached to a substrate and spacing a synthesized polymer from the substrate for exposure/binding to a receptor.

The term “solid support bound nucleotide” refers to a nucleic acid or an oligonucleotide that is covalently bonded to a solid-support. In all cases, the length of nucleotide(s) on a solid-support bound nucleotide is less than the length of nucleotides on a solid-support bound oligonucleotide that is produced from the solid-support bound nucleotide.

The terms “library of oligonucleotides” and “oligonucleotide array” are used interchangeably herein and refer to a collection of oligonucleotides which are produced in a single reaction apparatus.

The term “activator” refers to a compound that facilitates coupling of one nucleic acid to another, preferably in 3′-position of one nucleic acid to 5′- position of the other nucleic acid or vice a versa.

The terms “quality,” “performance” and “intensity” are used interchangeably herein when referring to oligonucleotide probes or binding of a target molecule to oligonucleotide probes mean sensitivity of oligonucleotide probes to bind to a target molecule while giving a minimum of false signals.

The terms “activated nucleoside” and “activated nucleotide” are used interchangeably herein in and refer to natural or unnatural nucleic acid monomers having a pendant activating group such as phosphite-triester, phosphotriester, H-phosphonate, or preferably phosphoramidite group on at least one of the oxygen atoms of the sugar moiety. Preferably, the activating group is on the C-3′ oxygen or C-5′ oxygen of the nucleic acid monomer. Typically, the activating group is on the C-3′ oxygen of the nucleic acid monomer, for synthesizing probes in the 3′-5′ direction, with the oligonucleotide attached to the support via the 3′-end. The activating group is on the C-5′ oxygen of the nucleic acid monomer, for synthesizing probes in the 5′-3′ (“reverse”) direction, with the oligonucleotide attached to the support via the 5′-end.

The terms “phosphoramidite,” “phosphoramidite derivative,” and “amidite” are used interchangeably herein and refer to a nucleic acid having a pendent phosphoramidite group.

The term “probe” refers to a surface-immobilized nucleic acid or oligonucleotide that is recognized by a particular target by virtue of having a sequence that is complementary to the target sequence. These may also be referred to as ligands.

The term “array” refers to a preselected collection of polymers which are associated with a surface of a substrate. In a preferred embodiment of the present invention, polymers are nucleic acids or, more preferably, oligonucleotide, which are also called oligonucleotide probes. An array can include nucleic acid or oligonucleotides of a given length having all possible monomer sequences made up of a specific basis set of monomers, or a specific subset of such an array. For example, an array of all possible oligonucleotides each having 8 nucleic acids includes 65,536 different sequences. However, as noted above, a nucleic acid or oligonucleotide array also can include only a subset of the complete set of probes. Similarly, a given array can exist on more than one separate substrate, e.g., where the number of sequences necessitates a larger surface area or more than one solid substrate in order to include all of the desired oligonucleotide sequences.

The term “wafer” generally refers to a substantially flat sample of substrate (i.e., solid-support) from which a plurality of individual arrays or chips can be fabricated.

The term “functional group” means a reactive chemical moiety present on a given monomer, polymer, linker or substrate surface. Examples of functional groups include, e.g., the 3′ and 5′ hydroxyl groups of nucleotides and nucleosides, as well as the reactive groups on the nucleobases of the nucleic acid monomers, e.g., the exocyclic amine group of guanosine, as well as amino and carboxyl groups on amino acid monomers.

The term photo protecting group (also called photo labile protecting groups or photo group for short) means a material which is chemically bound to a reactive functional group on a monomer unit, linker, or polymer and which may be removed upon selective exposure to electromagnetic radiation or light, especially ultraviolet and visible light.

The terms “array” and “chip” are used interchangeably herein and refer to the final product of the individual array of nucleic acid or oligonucleotide sequences, having a plurality of positionally distinct oligonucleotide sequences coupled to the surface of the substrate. “Array” is used with reference to nucleic acid or oligonucleotide, but it should be appreciated that either can be attached to a solid support. Reference will be made to oligonucleotide arrays as a preferred example of the present invention.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells. A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference. Additional methods of sample preparation and techniques for reducing the complexity of a nucleic sample are described in Dong et al., Genome Research 11, 1418 (2001), in U.S. Pat. No. 6,361,947, 6,391,592 and U.S. patent application Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. No. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

In accordance with one aspect of the present invention, a method is provided for preparing a high density oligonucleotide array on a solid support, the method comprising the following steps: a) providing a solid support having a surface comprising functional groups; b) attaching an activated nucleotide to a functional group in the presence of an activator; c) repeating the step of attaching an activated nucleotide to form an oligonucleotide array having at least 100 different oligonucleotides/cm².

In a preferred embodiment of the present invention, the array has at least 500 different oligonucleotides/cm². More preferably, the array has at least 1000 different oligonucleotides/cm². Still more preferably, the array has at least 5000 different oligonucleotides/cm². In most preferred embodiments of the present invention, the array has at least 10,000 different oligonucleotides/cm².

In a preferred embodiment of the present invention, the activated nucleotide is a phosphoramidite. More preferably, the phosphoramidite is located at the 3′ hydroxyl group of the nucleotide.

In a preferred embodiment of the present invention, the phorphoramidite further comprises a photo protecting group. Preferably, the photo protecting group is selected from the group consisting of NVOC, NPOC, MeNVOC, and MeNPOC. Most preferably, the photo protecting group is MeNPOC.

In a preferred embodiment of the present invention, the activator is selected from the group consisting of 4,5 dicyanoimidazole, 1-H-tetrazole, ethylthiotetrazole, a pyridinium salt, and mixtures thereof. More preferably, the activator is selected from the group consisting of ethylthiotetrazole, pyridinium trifluoroacetate, and mixtures thereof. Most preferably the activator is ethylthiotetrazole or pyridinium trifluoroacetate.

In one aspect of the present invention, a method is presented for preparing a high density oligonucleotide array on a solid support, said method having the following steps: a) providing a solid support having a surface comprising functional groups; b) attaching a phosphoramidite nucleotide to said functional group in the presence of ethylthiotetrazole; c) repeating said step of attaching an activated nucleotide to form an oligonucleotide array having at least 100 different oligonucleotides/cm².

The surface of the substrate is preferably provided with a layer of linker molecules, although it will be understood that the linker molecules are not required elements of the invention. See, e.g., U.S. Pat. No. 5,143,854, incorporated here by reference. The linker molecules are preferably of sufficient length to permit polymers in a completed substrate to interact freely with molecules exposed to the substrate. The linker molecules are preferably from 6-50 atoms long so as to provide sufficient exposure. The linker molecules may be, for example, aryl acetylene, ethylene glycol oligomers containing 2-10 monomer units, diamines, diacids, amino acids, or combinations thereof. Other linker molecules may be used in light of this disclosure.

According to one aspect of the present invention, linker molecules are selected based upon their hydrophilic/hydrophobic properties to improve presentation of synthesized polymers to certain receptors. For example, in the case of a hydrophilic receptor, hydrophilic linker molecules will be preferred so as to permit the receptor to more closely approach the synthesized polymer.

The linker molecules can be attached to the substrate via carbon-carbon bonds using, for example, (poly)trifluorochloroethylene surfaces, or preferably, by siloxane bonds (using, for example, glass or silicon oxide surfaces). Siloxane bonds with the surface of the substrate may be formed in one embodiment via reactions of linker molecules bearing trichlorosilyl groups. The linker molecules may optionally be attached in an ordered array, i.e., as parts of the head groups in a polymerized Langmuir Blodgett film. In alternative embodiments, the linker molecules are adsorbed to the surface of the substrate. The linker molecules and monomers used herein are provided with a functional group to which is bound a protective group. Preferably, the protective group is on the distal or terminal end of the linker molecule opposite the substrate. The protective group may be either a negative protective group (i.e., the protective group renders the linker molecules less reactive with a monomer upon exposure) or a positive protective group (i.e., the protective group renders the linker molecules more reactive with a monomer upon exposure). In the case of negative protective groups an additional step of reactivation will be required. In some embodiments, this will be done by heating.

The protective group on the linker molecules may be selected from a wide variety of positive light-reactive groups preferably including nitro aromatic compounds such as o-nitrobenzyl derivatives or benzylsulfonyl. In a preferred embodiment, 6-nitroveratryloxycarbonyl (NVOC), 2-nitrobenzyloxycarbonyl (NBOC) or alpha, alpha-dimethyl-dimethoxybenzyloxycarbonyl (DDZ) is used. Photo removable protective groups are described in, for example, Patchornik, J. Am. Chem. Soc. (1970) 92:6333 and Amit et al., J. Oro. Chem. (1974) 39:192, both of which are incorporated herein by reference.

In an alternative embodiment the positive reactive group is activated for reaction with reagents in solution. For example, a 5-bromo-7-nitro indoline group, when bound to a carbonyl, undergoes reaction upon exposure to light at 420 nm.

Generally, photolabile or photosensitive protecting groups include ortho-nitrobenzyl and ortho-nitrobenzyloxycarbonyl protecting groups. The use of these protecting groups has been proposed for use in photolithography for electronic device fabrication (see, e.g., Reichmanis et al., J. Polymer Sci. Polymer Chem. Ed. (1985) 23:1-8, incorporated herein by reference for all purposes).

Examples of additional photosensitive protecting groups which may be used in the light directed synthesis methods herein described, include, e.g., 1-pyrenylmethyloxycarbonyl, α,α-dimethyl-3,5-dimethoxybenzyloxycarbonyl, 4-methoxyphenacyloxycarbonyl, 3′-methoxybenzoinyloxycarbonyl, 3′,5′-dimethoxybenzoinyloxycarbonyl 2′,3′-dimethoxybenzoinyl-oxycarbonyl, 2′,3′-(methylenedioxy) benzoinyloxycarbonyl, N-(5-bromo-7-nitroindolinyl)carbonyl 3,5-dimethoxybenzyloxycarbonyl, and α-(2-methyleneanthraquinone)oxycarbonyl.

Particularly preferred photolabile protecting groups for protection of either the 3′ or 5′-hydroxyl groups of nucleotides or nucleic acid polymers include the o-nitrobenzyl protecting groups described in Published PCT Application No. WO 92/10092. These photolabile protecting groups include, e.g., nitroveratryloxycarbonyl (NVOC), nitropiperonyl oxycarbonyl (NPOC), alpha-methyl-nitroveratryloxycarbonyl (MeNVOC), alpha-methyl-nitropiperonyloxycarbonyl (MeNPOC), 1-pyrenylmethyloxycarbonyl (PYMOC), and the benzylic forms of each of these (i.e., NV, NP, MeNV, MeNP and PYM, respectively), with MeNPOC being most preferred.

Surprisingly and unexpectedly, the present inventors have found that by using the activators described herein, significantly less amounts of nucleotide phosphoramidites are required to achieve a high yield of high density solid support bound oligonucleotides. Therefore, the overall cost of producing high density solid-support bound oligonucleotide is substantially reduced.

In order to ensure efficiency and accuracy in synthesizing oligonucleotide arrays, it is generally desirable to provide a clean solid-support surface upon which the various reactions are to take place. Thus, methods of the present invention can also include a step of stripping the solid-support to remove any residual dirt, oils or other materials which may interfere with the synthesis reactions, or subsequent analytical use of the array.

The process of stripping the solid-support typically involves applying, immersing or otherwise contacting the solid-support with a stripping solution. Stripping solutions can be selected from a number of commercially available or readily prepared chemical solutions used for the removal of dirt and oils, which are well known to those skilled in the art. Particularly preferred stripping solutions are composed of a mixture of concentrated H₂SO₄ and H₂O₂. Such solutions are generally available from commercial sources, e.g., Nanostrip® from Cyantek Corp. After stripping, the solid-support is rinsed with water and in preferred aspects, is then contacted with a solution of NaOH, which results in regeneration of an even layer of hydroxyl functional groups on the surface of the solid-support. In some cases, the solid-support is again rinsed with water, followed by a rinse with HCl to neutralize any remaining base, followed again by a water rinse. The various stripping and rinsing steps can be carried out, for example, using a spin-rinse-drying apparatus of the type generally used in the semiconductor manufacturing industry.

Instead of a solution cleaning and preparation methods described above, gas phase cleaning and preparation methods can also be used. For example, by contacting the solid-support with H₂O or O₂ plasma or using reactive ion etching (RIE) techniques that are well known to one skilled in the art.

Following cleaning and stripping of the solid-support surface, the surface can be derivatized to provide alternative functional groups or linkers on the substrate or surface for synthesizing the various oligonucleotide sequences on that surface. See, e.g., U.S. Pat. Nos. 6,429,275, 6,410,675, 6,307,042, 5,959,098, and 5,919,523 each of which is incorporated here by reference. In particular, derivatization provides reactive functional groups, e.g., hydroxyl or amino groups or the like, to which the first nucleotides in the oligonucleotide sequence can be attached. In one aspect, the solid-support surface is derivatized using a silane in either water or ethanol or other organic solvent, or in the gas phase. Preferred silanes include mono- and dihydroxyalkyltrialkoxysilanes, which provide a hydroxyl functional group on the surface of the solid-support. Particularly preferred hydroxyalkyltrialkoxysilanes are N,N-bis(2-hydroxyethyl)aminopropyltriethoxysilane, N-(2-hydroxyethyl)-N-methyl-aminopropyltriethoxysilane, and N-(2-hydroxyethyl)-N,N-bis(triethoxysilylpropyl)amine.

Also preferred are aminoalkyltrialkoxysilanes which can be used to provide the initial surface modification with a reactive amine functional group. Particularly preferred are 3-3-aminopropyltrimethoxysilane (“APTMS”), aminopropyltriethoxysilane (“APTES”), and N,N′-bis(triethoxysilylpropyl)-1,2-diaminoethane and N,N-bis(triethoxysilylpropyl)amine. Derivatization of the solid-support using these latter amino silanes provides a linkage (e.g., phosphoramidate linkage) that is stable under synthesis conditions and final deprotection conditions.

In certain instances, amino silane derivatization provides several advantages over derivatization with hydroxyalkylsilanes. For example, the aminoalkyltrialkoxysilanes are inexpensive and can be obtained commercially in high purity from a variety of sources. Moreover, the resulting primary and secondary amine functional groups are more reactive nucleophiles than hydroxyl groups. Also, the aminoalkyltrialkoxysilanes are less prone to polymerization during storage. Furthermore, certain aminoalkyltrialkoxysilanes (“APTMS”, “APTES”), are sufficiently volatile to allow application in a gas phase in a controlled vapor deposition process.

Hydroxy groups in silanes can also be protected using suitable protecting groups to increase the stability or volatility of the silane. Such hydroxy protecting groups are well known to one of ordinary skill in the art. In most cases, silanes having protected hydroxy groups have higher vapor pressure (i.e., more volatile) than silanes having unprotected hydroxy groups. As such, silanes having hydroxy protecting groups can be readily purified by, e.g., distillation, and can be readily employed in gas-phase deposition methods of silanating solid-support surfaces. After coating these silanes onto the surface of the solid-support, the hydroxy groups can be deprotected, e.g., by a brief chemical treatment (e.g., dilute acid or base), which will not break the solid-support-silane bond, so that the solid-support can then be used for oligonucleotide synthesis. Examples of such silanes include, but are not limited to, acetoxyalkylsilanes, such as acetoxyethyltrichlorosilane, acetoxypropyltrimethoxysilane, which can be deprotected after application using, e.g., vapor phase ammonia or methylamine, or aqueous or ethanolic solution of ammonia or alkylamines. Epoxyalkylsilanes, such as glycidoxypropyltrimethoxysilane, can also be used. Such epoxyalkylsilanes can be deprotected using, e.g., vapor phase acid, or a dilute acid solution. Acetal protecting groups on hydroxyalkylsilanes can be similarly deprotected.

The physical operation of silanation of the solid-support generally involves dipping or otherwise immersing the solid-support in the silane solution. Following immersion, the solid-support is generally spun as described above for the solid-support stripping process, i.e., laterally, to provide a uniform distribution of the silane solution across the solid-support surface. This ensures a more even distribution of reactive functional groups on the solid-support surface. Following application of the silane layer, the silanated solid-support may be baked to anneal or stabilize the bonding of the silane to the solid-support surface. Baking typically takes place at temperatures in the range of from 90° C. to 120° C., preferably at 110° C., for a time period of from about 1 minute to about 10 minutes, preferably for about 5 minutes.

Alternatively, as noted above, the silane solution can be contacted with the solid-support surface using controlled vapor deposition methods or spray methods. These methods involve the volatilization or atomization of the silane solution into a gas phase or spray, followed by deposition of the gas phase or spray upon the solid-support surface, usually by ambient exposure of the solid-support surface to the gas phase or spray. Vapor deposition typically results in a more even application of the derivatization solution than simply immersing the solid-support into the solution.

The efficacy of the derivatization process, e.g., the density and uniformity of functional groups on the solid-support surface, can generally be assessed by adding a fluorophore which binds the reactive groups, e.g., a fluorescent phosphoramidite such as Fluoreprime® from Pharmacia, Corp., Fluoredite® from Millipore, Corp. or FAM® from ABI, and looking at the relative fluorescence across the solid-support surface.

General methods for the solid phase synthesis of a variety of polymer types, including oligonucleotides, have been previously described. Methods of synthesizing arrays of large numbers oligonucleotides on a single solid-support have also been described. See for example, U.S. Pat. Nos. 5,143,854, 5,384,261, 6,050,193, and PCT Publication No. WO 92/10092, all of which are incorporated herein by reference in their entirety. Oligonucleotide arrays may be fabricated as disclosed in, for example, U.S. Pat. Nos. 5,959,098 and 6,147,205, which are incorporated here by reference.

The synthesis of oligonucleotides on the solid-support surface can also be carried out using light directed methods. The light-directed or photolithographic synthesis methods involve a photolysis step and a chemistry step. The solid-support surface, prepared as described herein comprises functional groups on its surface. These functional groups are protected by photolabile protecting groups (i.e., are “photo protected”). During the photolysis step, portions of the solid-support surface are exposed to light or other activators to activate the functional groups within those portions, i.e., to remove photo protecting groups. The solid-support is then subjected to a chemistry step in which nucleotides that have at least one photo protected functional group are then contacted with the solid-support surface. These nucleotides covalently bond to the activated portion of the solid-support through an unprotected functional group.

Subsequent activation and coupling steps couple nucleotides to other portions, which can overlap with all or part of the first portion. The activation and coupling sequence at each portion on the solid-support determines the sequence of the oligonucleotide synthesized thereon. In particular, light is shone through the photolithographic masks which are designed and selected to expose, and thereby activate, a first portion of the solid-support. Nucleotides are then coupled to all or part of this portion of the solid-support. The masks used and nucleotides coupled in each step can be selected to produce arrays of oligonucleotides having a range of desired sequences, each sequence being coupled to a portion on the solid-support which location also dictates the oligonucleotide's sequence. The photolysis steps and chemistry steps are repeated until the desired sequences have been synthesized upon the solid-support surface.

Whether light directed methods or mechanical synthesis methods is used, oligonucleotide synthesis generally involves a coupling reaction (i.e., forming a covalent bond) between a nucleotide and an activated nucleotide derivative (e.g., a phosphoramidite derivative of nucleotide) in the presence of an activator. Solid phase oligonucleotide synthesis methods may employ tetrazole (“TET”) or DCI (4,5 dicyanoimidazole) as the activator and a nucleotide having a phosphoramidite functional group (i.e., amidite) as the activated nucleotide. While the yield of coupling reaction using TET or DCI is high in most cases, a high concentration of amidite is generally required. For example, to achieve a high coupling yield (e.g., >95%) of the coupled solid-support bound oligonucleotides, a standard solid phase oligonucleotide synthesis can require 100 mM amidite concentration. Lesser concentrations may be employed, however, such lesser concentrations may require a longer reaction time. Amidite concentrations that may be used in conjunction with the present invention include for example 1 to 100 mM. Preferably, amidite concentrations are less than 50 mM. In the synthesis of DNA arrays on a planar support, application of the activated amidite can require a minimum volume (several mls) in order to reliably cover the entire surface of the wafer. Since the total quantity of surface reaction sites is relatively small, there is an excess of the activated amidite in solution so that much of it remains unreacted after the coupling reaction is completed, and subsequently goes to waste. Thus, to conserve reagent costs, it is important to reduce amidite concentration to a minimum, but still allow completion of the coupling reaction in a reasonable amount of time.

As stated above, it has been found in accordance with one aspect of the present invention that by using the activator disclosed herein in conjunction with the fabrication of high density arrays, the amount of activated nucleic acid required to achieve a high coupling reaction yield can be significantly reduced. Moreover, in accordance with one aspect of the present invention, the quality of the oligonucleotides produced on high density arrays by methods of the present invention is also significantly higher than standard coupling methods.

Activator concentrations that may be used in accordance with the present invention are from 1 to 1000 mM. More preferably, activator concentrations are from 50 to 500 mM.

In one embodiment, methods of the present invention are used in a flow cell reactor system. Such reactor systems are particularly suited for the combined photolysis/chemistry process as generally described in the above mentioned commonly assigned U.S. Pat. Nos. 5,424,186 and 5,959,098. A schematic illustration of a device for carrying out the combined photolysis/chemistry steps of the individual process can also be found in the above mentioned U.S. Patents.

Briefly, in a flow cell reactor system, the solid-support is mounted in a flow cell during both the photolysis and chemistry or monomer addition steps. In particular, the solid-support mounted in a reactor system that allows for the photolytic exposure of the synthesis surface of the solid-support to activate the functional groups thereon. Solutions containing appropriate reagents (e.g., amidite having a photo protected hydroxy group and the activator) are then introduced into the reactor system and contacted with the synthesis surface (e.g., functional group on the solid-support, linker, or solid-support bound nucleotide), where the activated nucleotide can bind with the active functional groups (e.g., free hydroxyl groups) on the solid-support surface. For example, where the synthesis is in the 3′ to 5′ direction of oligonucleotide, a solution containing a 3′-O-activated phosphoramidite nucleoside, photo protected at the 5′-hydroxyl is introduced into the flow cell for coupling to the photo activated regions of the solid-support. Preferably, separate solutions of the activated nucleotide (e.g., amidite) and the activator are introduced simultaneously or sequentially to the reactor system.

The activated nucleotide can be dissolved in any inert solvent including acetonitrile, tetrahydrofuran, dimethylsulfoxide, dioxane, dichloromethane, nitromethane, dimethyl formamide, toluene, propylene carbonate, and combinations thereof. Typically, however, the activated nucleotide is dissolved in acetonitrile.

The activator can be dissolved in any inert solvent including acetonitrile, tetrahydrofuran, dimethylsulfoxide, dioxane, dichloromethane, nitromethane, dimethyl formamide, toluene, propylene carbonate, and combinations thereof. Typically, however, the activator is dissolved in acetonitrile.

In a flow cell reactor system, the coupling reaction temperature range is typically from about 15° C. to about 35° C., preferably from about 20° C. to about 30° C., and more preferably from about 20° C. to about 25° C. In one embodiment, the coupling reaction temperature is about 25° C. or less.

The amount of total activated amidite used during each coupling step is generally from about 4 mM concentration to about 12 mM in the reactor system, preferably from about 5 mM to about 8 mM, and more preferably from about 6 mM to about 7 mM.

Typical the coupling reaction is allowed to proceed for from about 5 seconds to about 5 minutes. Preferably, the coupling reaction time allowed to proceed for from about 15 seconds to about 60 seconds, and more preferably from about 10 to about 30 seconds. The coupling reaction should be allowed to proceed until no further significant coupling reaction occurs to maximize the yield of coupled product. Such coupling reaction can be monitored using any of the reaction monitoring methods known to one of ordinary skill in the art, e.g., thin-layer chromatography, gas chromatography, HPLC, spectral analysis of one or more starting materials (e.g., UV, IR, NMR, etc.), and the like.

After the coupling reaction, the resulting solution which may comprise the amidite and/or the activator is then removed from the reactor system (e.g., by washing with a solvent). The wafer is then rinsed, e.g., with acetonitrile, and then standard capping and oxidation steps are performed. Another photolysis step is then performed, exposing and activating different selected regions of the solid-support surface. This process is repeated until the desired oligonucleotide arrays are created.

During each photolysis step, the solid-support can be irradiated either in contact or not in contact with a solution and is, preferably, irradiated in contact with a solution. Preferably, the solution comprises reagents to prevent the by-products formed by irradiation from interfering with synthesis of oligonucleotides. Such by-products can include, for example, carbon dioxide, nitrosocarbonyl compounds, styrene derivatives, indole derivatives, and products of their photochemical reactions. Alternatively, the solution can comprise reagents used to match the index of refraction of the solid-support. Reagents added to the solution can further include, for example, acidic or basic buffers, thiols, substituted hydrazines and hydroxylamines, reducing agents (e.g., NADH) or reagents known to react with a given functional group (e.g., aryl nitroso+glyoxylic acid→aryl formhydroxamate+CO₂).

The added activated nucleotide typically includes a single active functional group, for example, a phosphoramidite on the 3′-hydroxyl group. The remaining functional group that is involved in linking the activated nucleotide within the oligonucleotide sequence, e.g., the 5′-hydroxyl group of a nucleotide, is generally protected (e.g., with a photolabile protecting group). The activated nucleotides then bind to the reactive moieties on the surface of the solid-support, activated during the preceding photolysis step, or at the termini of linker molecules, nucleotides, or oligonucleotides being synthesized on the substrate.

The use of activators in solid phase synthesis of RNA and DNA has been described. See, e.g., Annovis Technical Bulletin, No. 56, September 2001, Annovis, Inc., 34 Mount Pleasant Drive, Aston, Pa. 19014, Tel. 610-361-9224, fax: 610-361-8255, www.annovis.com, incorporated here by reference (5-Ethylthio-1H-tetrazole as an activator in Oligonucleotide Synthesis). Annovis reports that ETT is a highly efficient activator for both RNA and oligonucleotide synthesis. See also Wincott, F. et al., Nucleic Acids Res., 1995, 23, 2677; Sproat, B. et al., Nucleoside Nucleotide, 1995, 14, 1481; Tsou, D. et al., Nucleoside Nucleotide, 1995, 14, 1481; Wright, P., et al., Tet Lett, 1993, 34, 3373, all of which are incorporated here by reference. Pyridinium trifluoro acetate is also know to be an efficient activator for oligonucleotide synthesis. See, e.g., Eleuteri, A., Capaldi, D. C., Krotz, A. H., Cole, D. L., & Ravikumar, V. T. (2000) Pyridinium trifluoroacetate/N-methylimidazole as an efficient activator for oligonucleotide synthesis via the phosphoramidite method. Organic Process Research and Development, 4, 182-189. #15021.

### EXAMPLES

### Example 1

Chart 1 shows the hybridization performance of a control checkerboard array vehicle synthesized using different activators and probed with a control probe. The data indicate that using either ETT or PTA as activators provides a 10-20% improvement in hybridization signal intensity compared to DCI or TET, while maintaining equivalent intra- and inter-wafer uniformity. The activator concentrations used were DCI, 125 mM; TET, 225 mM; ETT, 125 mM; and PTA, 250 mM.

[image]

### Example 2

Chart 2 shows the hybridization performance of a human full length (HFL) gene expression product arrays synthesized using different activators and probed with an appropriate probe. The data indicate that using either ETT or PTA as activators provides a 20-30% improvement in hybridization signal intensity compared to DCI or TET, while maintaining equivalent intra- and inter-wafer uniformity. The activator concentrations used were DCI, 125 mM; TET, 225 mM; ETT, 125 mM; PTA, 250 mM.

[image]

### Example 3

Chart 3 shows a comparison of activators used in the synthesis of a control checkerboard array test vehicle, in terms of array hybridization intensity, after reducing the amidite concentration used in Examples 1 and 2 to half. The activator concentrations used were DCI, 125 mM; TET, 225 mM; ETT, 125 mM; PTA, 250 mM. This experiment was performed to determine whether amidite consumption could be reduced while maintaining acceptable synthesis yield and hybridization performance. The results indicate that acceptable performance was maintained at lower amidite concentration using DCI, ETT, and PTA.

[image]

### Example 4

Chart 4 shows a comparison of activators used in the synthesis of Human—Full Length (hfl) gene expression product arrays, in terms of array hybridization intensity, after reducing the amidite concentration to half that used in Examples 1 and 2. The activator concentrations used were DCI, 125 mM; TET, 225 mM; ETT, 125 mM; PTA, 250 mM. This experiment was performed to determine whether amidite consumption could be reduced while maintaining acceptable product performance. The results indicate that acceptable or better performance was maintained at the lower amidite concentration using DCI, ETT, and PTA. A marked improvement in hybridization average difference was noted for the arrays synthesized with ETT and PTA relative to DCI.

[image]

### Example 5

Chart 5 shows a comparison of activators used in the synthesis of checkerboard test vehicle arrays, in terms of array hybridization intensity, after further reducing the amidite concentration used in the coupling step to quarter of that used in Examples 1 and 2. The activator concentrations used were DCI, 125 mM; TET, 225 mM; ETT, 125 mM; PTA, 250 mM. This experiment was performed to determine whether amidite consumption could be further reduced while maintaining acceptable synthesis yield and hybridization performance. The results indicate that acceptable performance was maintained at lower amidite concentration using DCI and ETT.

[image]

The foregoing discussion of the invention has been presented for purposes of illustration and description. The foregoing is not intended to limit the invention to the form or forms disclosed herein. All references cited herein are incorporated herein by reference in their entirety. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for preparing a high density oligonucleotide array on a solid support, said method comprising the following steps:
a) providing a solid support having a surface comprising functional groups;
b) attaching an activated nucleotide to said functional group in the presence of an activator;
c) repeating said step of attaching an activated nucleotide to form an oligonucleotide array having at least 100 different oligonucleotides/cm².

2. A method for preparing a high density oligonucleotide array according to claim 1 wherein said array has at least 500 different oligonucleotides/cm².

3. A method for preparing a high density oligonucleotide array according to claim 1 wherein said array has at least 1000 different oligonucleotides/cm².

4. A method for preparing a high density oligonucleotide array according to claim 1 wherein said array has at least 5000 different oligonucleotides/cm².

5. A method for preparing a high density oligonucleotide array according to claim 1 wherein said array has at least 10,000 different oligonucleotides/cm².

6. A method according to claim 1 wherein said activated nucleotide is a phosphoramidite.

7. A method according to claim 1 wherein said phosphoramidite is located at the 3′ hydroxyl group of a nucleotide

8. A method according to claim 6 wherein said phorphoramidite further comprises a photo protecting group.

9. A method according to claim 8 wherein said photo protecting group is selected from the group consisting of NVOC, NPOC, MeNVOC, and MeNPOC.

10. A method according to claim 9 wherein said photo protecting group is MeNPOC.

11. A method according to claim 1 wherein said activator is selected from the group consisting of 4,5 dicyanoimidazole, 1-H-tetrazole, ethylthiotetrazole, a pyridinium salt, and mixtures thereof.

12. A method according to claim 1 wherein said activator is selected from the group consisting of ethylthiotetrazole, pyridinium trifluoroacetate, and mixtures thereof.

13. A method according to claim 1 wherein said activator is ethylthiotetrazole.

14. A method according to claim 1 wherein said activator is pyridinium trifluoroacetate.

15. A method for preparing a high density oligonucleotide array on a solid support, said method comprising the following steps:
a) providing a solid support having a surface comprising functional groups;
b) attaching a phosphoramidite nucleotide to said functional group in the presence of ethylthiotetrazole;
c) repeating said step of attaching an activated nucleotide to form an oligonucleotide array having at least 100 different oligonucleotides/cm².
